(19) United States
(12) Patent Application Publication
(10) Pub. No.: US 2017/0367692 A1
(43) Pub. Date: Dec. 28, 2017

(54) SYSTEM, METHOD AND APPARATUS FOR TENORRHAPHY

(71) Applicant: Flexios LLC, West University Place, TX (US)

(72) Inventors: Michael W. DEEM, West University Place, TX (US); Kimberly M. NEELY, Houston, TX (US); Forrest S. ROTH, Houston, TX (US)

(21) Appl. No.: 15/636,271

(22) Filed: Jun. 28, 2017

Related U.S. Application Data
(60) Provisional application No. 62/355,674, filed on Jun. 28, 2016.
(60) Provisional application No. 62/385,677, filed on Sep. 9, 2016.

(51) Int. Cl. A61B 17/04; A61B 17/062; A61B 17/06; A61B 17/00

(52) U.S. Cl. CPC A61B 17/0483; A61B 17/0466; A61B 17/0467; A61B 17/0482; A61B 17/06004; A61B 17/06166; A61B 17/062; A61B 17/0401; A61B 2017/0023; A61B 2017/00367; A61B 2017/00893; A61B 2017/0464; A61B 2017/06076; A61B 2017/06176; A61B 2017/00004

(57) ABSTRACT

The invention comprises a system, method, and apparatus for securing two ends of a ruptured or partially ruptured tendon in the hand or wrist during a repair, recession, or revision procedure. During this tenorrhaphy, anchoring material is inserted into the proximal ruptured surface of the tendon by an apparatus comprising multiple needles of at least two different types. Connecting material spans the site of tenorrhaphy of a ruptured tendon, or a multiplicity of ruptured tendons. In some embodiments, interlocking patterns of loop and helical suture are inserted by straight and helical needles, respectively.

### RELATED U.S. APPLICATION DATA

The benefit of U.S. Provisional Application No. 62/355,674 filed Jun. 28, 2016, and of U.S. Provisional Application No. 62/385,677 filed Sep. 9, 2016, is hereby claimed, each of which along with its subject matter is incorporated by reference herein, as if it had been set forth hereinafter in its entirety.

### REFERENCES

The following patent and other documents (along with their subject matter) are incorporated by reference herein, as if fully set forth hereinafter:

### U.S. Patent Documents

| | | |
|---|---|---|
| 6,083,244 | July 2000 | Lubbers et al. |
| 6,537,248 | March 2003 | Mulier et al. |
| 6,626,917 | October 2003 | Craig |
| 7,637,918 | December 2009 | Dant |
| 7,776,059 | August 2010 | Craig |
| 8,114,129 | February 2012 | Lubbers et al. |
| 8,313,013 | November 2012 | Kuester, III et al. |
| 8,500,757 | August 2013 | Miraki et al. |
| 8,795,333 | August 2014 | Gordon et al. |
| 8,906,039 | December 2014 | Crainich |
| 8,968,334 | March 2015 | Ostrovsky et al. |

### Other Documents

Tendon Surgery of the Hand, Edited by Jin Bo Tang, Peter C. Amadio, Jean Claude Guimberteau, and James Chang, 2012, ISBN 978-1437722307 (Elsevier, Oxford).
Caulfield et al., “Comparison of Zones 1 to 4 Flexor Tendon Repairs Using Absorbable and Unabsorbable Four-Strand Core Sutures”, (The Journal of Hand Surgery, Euro. Vol. 2008. Vol. 33E:412-417)
Kang et al., “Flexor Tenorrhaphy Using Absorbable Suture Materials”, (Archive of Plastic Surgery, May 23, 2012, Vol. 39:397-403)
Khor et al., “Improving Outcomes in Tendon Repair: A Critical Look at the Evidence for Flexor Tendon Repair and Rehabilitation”, (Plastic and Reconstructive Surgery Journal, 2016. Vol. 138:1045e)
Marrero-Amadeo et al., “Flexor Tendon Repair with a Knotless Barbed Suture: A Comparative Biomechanical Study”, (Journal of Hand Surgery, 2011. Vol. 36A:1204-1208)
Parikh et al., “Barbed Suture Tenorrhaphy; An Ex Vivo Biomechanical Analysis”, (Plastic and Reconstructive Surgery, November 2009. Vol. 124: 1551-1558)
Shrewsbury and Kuczynzki, “Flexor Digitorum Superficialis Tendon in the Finger of the Human Hand”, (The Hand, 1974. Vol. 6, No. 2:121-133)
Sull et al., (American Association for Hand Surgery 2015 Annual Meeting, “Does Barbed Suture Repair Negate the Benefits of Peripheral Repair in Porcine Flexor Tendon?”, No. 2, p.2)
Strickland, (Journal of the American Academy of Orthopaedic Surgeons, 1995. 3:44-54).
Tan et al., “Animal models for training in tendon surgery: sheep's forelimbs”, (Journal of Hand Surgery, February 2010. Vol. 35E152-153).
Walbeehm and McGrouther, “An Anatomical Study of the Mechanical Interactions of Flexor Digitorum Superficialis and Profundus and the Flexor Tendon Sheath in Zone 2”, (Journal of Hand Surgery, British & European Vol. 1995., Vol. 20B:269-280)

### FIELD OF THE INVENTION

This invention relates to tenorrhaphy: the repair, recession, and revision of tendon ruptures, and especially hand and wrist tendon ruptures, to simulate the original orthopedic structure or to accomplish a remodeling of such structure. Practice of the invention will increase repaired tendon strength, increase favorable patient outcome, reduce surgical procedure time, or allow earlier tendon flexion after surgery.

### BACKGROUND OF THE INVENTION

Rupture of tendons, especially in the hand and wrist, is a common acute injury. Approximately 100,000 corrective procedures are performed in the United States annually to repair ruptured or partially ruptured tendons in the hand and wrist (Tang et at, 2012). This corrective procedure is known as tenorrhaphy. Flexor digitorum tendons have a typical cross-sectional dimension of 3-8 mm (Walbeehm and McGrouther, 1995; Shrewsbury and Kuczynzki, 1974). When a significant amount of tendon has been lost in the acute injury, graft tendon material is used to reform the ruptured tendon, in what is termed a recession surgical procedure. By far the most common surgical expedient for joining tendons is a suture, and among the many suture techniques are the modified Kessler, Stevens, and cruciate procedures. In 2-4% of repairs, there is total failure of the suture (Tang et al., 2012). In another 20-30% of cases, there is poor to fair outcome of the repair procedure, as measured by the Strickland scale (Tang et al., 2012; Strickland, 1995). A second, revision surgical procedure can be used in the rupture cases and in some of the failed or poor to fair outcome cases to improve the result. Sheep's forelimb digital flexor tendons are described as a good model of human flexor digitorum tendons (Tan et al., 2010).

There are a number of reasons for the limitations of current surgical procedures. Among the causes of poor outcome are weakness in proximity of the repaired tendon rupture and excessive scarring and adhesion of the tendon to sheathing, tunnels, or pulleys. The repair is often very weak before the tendon heals, and what would otherwise be normal movement in a healthy tendon may cause another rupture tendon repaired via suture. The body part in question is often immobilized for a significant amount of time; for instance, the hand or wrist of the patient is typically immobilized for 6-8 weeks following the surgery. This immobilization, while protecting the joined tendon from rupture, inhibits recruitment of new blood flow and tissue to the wound. The immobilization also leads to the formation of scar and other tissue that can adhere the tendon to the surrounding matrix, leading to joint, stiffness and poor long term prognosis. Indeed, early active mobilization remains the only long-term, proven strategy to improve outcomes (Khor et al. 2016). Such early mobilization requires a strong repair.

Absorbable suture has been proven suitable for some types of tendon repair. Caulfield et al. (2008) reported the outcome of flexor tendon injuries in a series of 272 consecutive patients treated over 45 months with a mean follow-up of 4 (range 3-12) months. Using the original Strickland criteria, there were 72 excellent/good and 28% fair/poor results in the absorbable core suture group, and 73% and 27%, respectively, in the nonabsorbable core suture group. This study suggested that appropriate absorbable core sutures can be used safely for flexor tendon repair. Kang et al. (2012) likewise showed in a study of flexor tendon repairs with absorbable suture that all the patients were assessed to be excellent or good using the original Strickland criteria. Also, fibrosis and long-term foreign body tissue reactions such as stitch granuloma were less likely to occur in their study. Compared to the Cullen's report as a reference for data on performance of nonabsorbable sutures, there was no significant difference in the rupture or adhesion rates. Therefore, this study, suggests that appropriate absorbable core sutures can be used safely for flexor tendon repairs.

Parikh et al. (2009) reported, on an ex vivo model of flexor tenorrhaphy. A three-strand barbed suture technique achieved tensile strength comparable to that of four-strand cruciate repairs and demonstrated significantly less repair-site bunching. A six-strand barbed suture technique demonstrated increased tensile strength compared with four-strand cruciate controls and significantly less repair-site bunching. They concluded that barbed suture repair may offer several advantages in flexor tenorrhaphy.

Marrero-Amadeo et al. (2011) tested whether a flexor tendon repair with only a knotless barbed suture technique provides a repair with a greater maximal load to failure and 2-mm gapping resistance than a traditional technique using a 4-strand core plus a running-locking epitendinous suture. The average maximal load to failure was not significantly different between the traditional repair (48+/−12 N) and the barbed suture repair (50+/−14 N). The average 2-mm gapping load was also insignificantly different between the traditional repair (42+/−12 N) and the barbed suture repair (32+/−9 N). The barbed suture repair did not demonstrate a significant difference in maximal load to failure and 2-mm gapping resistance compared with the traditional method of repair. A porcine model showed no difference between barbed and barbless repairs in a porcine flexor tendon model (Sull et al, 2015).

There exist a number of prior approaches to tendon repair. Lubbers et al. U.S. Pat. No. 6,083,244 describes an elongate tensile member to which anchor bodies are attached. The anchor bodies are inserted into the tendon via a wound in the tendon. The anchor bodies are slid long the tensile member to adjust the length of repair. Lubbers et al. U.S. Pat. No. 8,114,120 describes an apparatus and methods for repairing damaged tendons or ligaments. Various repair apparatus include an elongate tensile member and a pair of anchor assemblies connected for movement along the tensile member on either side of a repair site, such as a tear or laceration. A soft tissue anchor is coupled to the tensile member outside of the tendon. Alternatively, the assembly of the soft tissue anchor connected to the tensile member is simultaneously driven into the tendon, in which case the soft tissue anchor includes a helical anchor member and a retaining member. Ostrovsky at al. U.S. Pat. No. 8,968,334 describes apparatus that includes an elongate member having a distal end portion and a proximal end portion The elongate member is used to drive suture'into the tendon. Tissue anchors are attached to the suture during the insertion process.

Dant U.S. Pat. No. 7,637,918 describes an apparatus for repairing, a tear in an annulus fibrosus of a spinal disc that includes a hollow, helically-shaped suturing needle and a retriever. The helical suture pattern spans both sides of the tear, such that the loops of the helix bridge the tear. Similarly, Crainich U.S. Pat. No. 8,906,039 describes a suturing device that includes a housing, a helical needle rotatably mounted relative to the housing, and a drive assembly for rotating the needle relative to the housing whereby the helical needle carries a suture through tissue to be sutured. The helical suture pattern spans both sides of the torn tissue, such that the loops of the helix bridge the tear. Similarly, Craig U.S. Pat. No. 7,776,059 describes an apparatus used with a helical suture device that has a first end and a second end. The helical suture pattern spans both sides of the torn tissue, such that the loops of the helix bridge the tear. And, Craig U.S. Pat. No. 6,626,917 describes a helical, suturing instrument that either pushes or pulls a suture along a helical needle tract. The instrument has one or more helical needle portions, each with a suture retaining component.

Gordon et al. U.S. Pat. No. 8,795,333 describes a method and apparatus for reattaching the opposed ends of a member, such as a tendon, ligament or bone, during preparing and healing of the member using a surgical repair device that can be securely attached to the member and then safely guided through tortuous anatomy for reattachment and repair. All require retrieving through an anatomical passage a first stump of a longitudinal anatomical feature for reattachment to another anatomical feature, and a single type of suture pattern is used to attach the repair to the end of the ruptured tendon, with breach of epitenon.

Miraki et al. U.S. Pat. No. 8,500,757 describes a suture applicator having a proximal handle, a length of suture wound on a spool, and a hollow suture needle extending from a distal end of the handle. The hollow needle can be guided by a mechanism such as a deflection wire.

Kuester, Ill et al. U.S. Pat. No. 8,313,013 describes an assembly for anastomosis having a clamp being arranged to support a fastener. This device is for coupling veins rather than tendons and does not employ suture.

Muller et al. U.S. Pat. No. 6,537,248 describes a surgical apparatus for delivering a helical pattern of conductive fluid to a target site for subsequent formation of a virtual electrode to ablate bodily tissue at the target site by applying a current to the delivered conductive fluid.

### SUMMARY OF THE INVENTION

The present invention comprises in various aspects a novel system, method and is apparatus for securing two ends of ruptured or partially ruptured tendon in the hand or wrist during a repair, recession or revision procedure. With the present inventive system and method, a connector, such as suture material, is inserted into or affixed in one of proximal ends of the completely or partially ruptured tendon whereby it is positioned such that another part of said connector can be attached to a further one of said proximal ends across the site of the rupture. This is accomplished by an innovative apparatus comprising multiple needles of at least two different types. In such manner, the connector (e.g., suture material) can be secured across the site of tenorrhaphy for a ruptured tendon as aforesaid.

In one aspect, the invention is in a system for the repair of a ruptured tendon, which comprises a plurality of high surface area anchor, members each such member being operative for implantation in a part of the tendon proximal the rupture, and each such member further being adapted for interconnection with another such member also operative for implantation in a part of the tendon proximal the rupture; and a connector for attaching one of said members to one another of said members.

In another aspect, the invention is in a method for repair of a ruptured tendon, which comprises implanting a plurality of high surface area anchor members in respective different parts of the tendon proximal the rupture; and interconnecting one of said members with another of said members.

In yet another aspect, the invention is in an apparatus for implanting a repair assembly including a plurality of high surface area anchor members into a ruptured portion of a tendon, which comprises a needle assembly for implanting an anchor member as aforesaid in a part of the tendon proximal said rupture, and for attaching one part of a connector to one of said anchor members whereby it is positioned for attaching of another part thereof to another anchor member.

In still another aspect, the invention a method for deploying a connector adapted to secure two ends of a ruptured (completely or partially) tendon, which comprises: introducing multiple needles of at least two different types which are operative to attach a connector, such as suture material, to a proximal cut surface of the tendon; and attaching a part of the connector to said proximal cut surface with said needles.

In further aspects, the invention is in an apparatus, which comprises multiple needles of at least two different types operative to attach a connector, such as suture material, to a proximal cut surface of the tendon.

In a still further aspect, the invention is in a system for the repair of a ruptured tendon, which comprises a first complement of at least two differently patterned sutures implanted by needles of two or more different types in a part of the tendon proximal the rupture, each such patterned suture further being intertwined with another of such differently patterned sutures; a second complement of at least two differently patterned sutures implanted in another part of the tendon proximal to such rupture, each such patterned, suture of the second complement being intertwined with another of such second complement's differently patterned sutures, such first and second suture complements being interconnected with one another.

Moreover, the invention is in a method for repair of a ruptured tendon, which comprises implanting a first complement of at least two differently patterned sutures in the tendon proximal the rupture and a second such suture complement in a different part of the tendon proximal the rupture; and interconnecting said first and second suture complements with one another.

And, in yet other aspects, the invention is in apparatus and method for inserting a repair system including a first suture into a ruptured portion of a tendon, which comprises an assembly for implanting (or the step of implanting) a patterned suture as aforesaid in a part of the tendon proximal said rupture, and an assembly for introducing (or the step of introducing) a separate (optionally differently patterned) suture for connecting said implanted first suture material across the site of tenorrhaphy to a different part of the tendon.

The term “suture” as used herein refers inclusively to the article utilized to join tendon (or other body) tissues to one another (for instance, thread, wire, or other comparable functional form), pieces or other elements of such article, and the constituent material thereof. The meaning intended concerning any particular appearance of the term shall be as appropriate to the context.

An advantage of practicing the invention is greater strength of the joined tendon. The greater strength is achieved by the high surface area to volume ratio of the patterned sutures employed. This greater strength leads to less likelihood of a failed surgical procedure.

Another advantage of the present inventive approach is the earlier, possibly immediate, flexion of the tendon after surgery. Said flexion increases recruitment of tissue and blood flow to the site of the injury, speeding remodeling. Said flexion also reduces inappropriate adhesion of the tendon to the surrounding sheathing, pulleys, and tunnels.

Yet another advantage of the present inventive approach is a smoother tendon repair or revision. A smooth, tubular tendon joint allows for smoother gliding of the tendon through pulleys and tunnels.

Still another advantage of the present inventive approach is a more flexible tendon joint, which allows for smoother gliding of the tendon through pulleys and tunnels. As regards hand tendons specifically, a more flexible tendon joint allows for a wider range of motion of the affected fingers, as flexion can cause high curvature of the tendon over joints and pulleys, which is better accommodated by a flexible joint.

A further advantage of the present invention is minimal required exposure of the tendon at the site of the acute injury. Unlike sutures wherein at least 1 cm of tendon distal to the site of acute injury must be exposed in the present method only enough tendon for a secure grip during implantation of patterned suture material during surgery is required. Thus the present invention requires a significantly smaller flap of skin, tunnel, and pulley material to be exposed.

Additional advantages of the invention are increased levels of patient care and satisfaction, as well as attainment of a more rapid and reproducible procedure. A faster procedure allows for more efficient utilization of surgical and facility resources. Reproducibility and standardization of the procedure are important factors in achieving a good or excellent outcome. Reproducibility and standardization lead to a more predictable outcome, a desirable feature for the medical profession and insurance market.

These, and other embodiments and advantages that will appear from the invention's description, imply a higher likelihood of good long term performance in the patient. The patient will experience less pain and a more rapid recovery. Moreover, it is more likely that only a single surgery will be required.

### BRIEF DESCRIPTION OF THE DRAWINGS

FIG. 1 provides a side view of an assembly containing disposable and reusable parts, in accordance with an embodiment of the invention.

FIG. 2 provides a side view of an assembly in which disposable needle parts are contained within a disposable cartridge, which can be located within a tool body, in accordance with an embodiment of the invention.

FIG. 3 provides an axonometric view of an assembly in which disposable needle parts are located within a tool body in accordance with an embodiment of the invention.

FIG. 4 provides a side view of forceps holding a ruptured tendon and a clamp element in accordance with an embodiment of the invention adapted to provide resistance during needle insertion.

FIG. 5 provides a side view of the clamp element during engagement with tendon n accordance with an embodiment of the invention.

FIG. 6 provides a transparent top view of an interlocking suture pattern deployed within the tendon in accordance with an embodiment of the invention.

FIG. 7 provides a section view of helical and straight needles contained within a needle assembly in accordance with an embodiment of the invention.

FIG. 8 provides a section view of straight needles and suture in the tendon at a point midway through the deployment process in accordance with an embodiment of the invention.

FIG. 9 provides a section view of suture and a helical needle having a groove and a hole adapted to hold barbed suture, in the tendon at a point midway through the deployment process in accordance with an embodiment of the invention.

FIG. 10 provides a view of a gate sub-assembly adapted to facilitate the alignment of the needles utilized according to an embodiment of the invention.

FIG. 11 provides (to the left) a front view of a first set of disposable needle parts within the tool body, and (to the right) a front view of second set of disposable needle parts brought to bear via a shift within the tool body in accordance with an embodiment of the invention.

FIG. 12 provides an axonometric view of a pair of straight needles attached to a sliding member in accordance with an embodiment of the invention.

FIG. 13 provides a side view of a straight needle having a notch adapted to hold suture accordance with an embodiment of the invention.

FIG. 14 provides a side view of loop suture engaged in a notch of a straight needle in accordance with an embodiment of the invention.

FIG. 15 provides an axonometric view of the sharpened tip and notch of a straight needle and a section view of the sharpened tip in accordance with an embodiment of the invention.

FIG. 16 provides top views of straight needles having notches adapted to hold suture, and suture within the tendon at various stages, (from left to right) as the needle is withdrawn in accordance with an embodiment of the invention.

FIG. 17 provides an axonometric view of a helical needle attached, to a tube in, accordance with an embodiment of the invention.

FIG. 18 provides an axonometric view of a helical needle having a groove and hole adapted to retain suture and a section view of the sharpened tip in accordance with an embodiment of the invention.

FIG. 19 provides axonometric views of the sharpened reverse-cutting tip, groove, and hole of the helical needle in accordance with an embodiment of the invention.

FIG. 20 provides an axonometric view of barbed suture engaged with a helical needle having a groove and hole adapted to retain suture in accordance with an embodiment of the invention.

FIG. 21 provides an axonometric view of smooth suture engaged with a helical needle having a groove and hole adapted to retain suture in accordance with an embodiment of the invention.

FIG. 22 provides transparent top views of a helical needle and suture within the tendon at various stages (from left to right) as the helical needle is withdrawn in accordance with an embodiment of the invention.

FIG. 23 provides a transparent top view of interlocking suture patterns deployed within the tendon to facilitate tenorrhaphy in accordance with an embodiment of the invention.

FIG. 24 provides a transparent top view of interlocking suture patterns deployed within the tendon, fastened and tightened across the site of tenorrhaphy with multiple knots to adjoin the tendon ends, in accordance with an embodiment of the invention.

FIG. 25 provides a transparent top view of suture fastened across the site of tenorrhaphy to approximate the severed tendon ends in which the interlocking pattern has been set, in accordance with an embodiment of the invention.

FIG. 26 provides a transparent top view of two instances of loop and helical suture pattern repairs fastened across the site of tenorrhaphy to approximate the severed tendon ends in accordance with an embodiment of the invention.

FIG. 27 provides a top view of a knot operative to facilitate the adjustment of the length of the suture in accordance with an embodiment of the invention.

FIG. 38 shows a high surface area attachment, where multiple barbs provide the engagement force on the tendon.

### DETAILED DESCRIPTION OF CERTAIN EMBODIMENTS OF THE INVENTION

The following detailed description is meant to be read not only in its stand-alone form, but also (as helpful) with reference to the figures. The figures, which are not necessarily to scale, depict selected embodiments and are not intended to limit the scope of the invention. Examples of construction, material, dimension, and manufacturing process particulars are provided for selected elements. All other elements employ that which is known to those of skill in the field of the invention. Those skilled in the art will recognize that many of the examples provided have suitable alternatives that can be utilized.

The invention (especially the inventive system, method and apparatus and other embodiments) is, for instance, beneficial for repairing ruptured hand or wrist tendons. As regards hand tendons, we have in mind especially tendons in zone 2 in the hand, such as flexor tendons in zone 2 of the hand.

In respect of the system, method, apparatus and other embodiments of the invention, an anchor assembly can be a simple double-sided high-surface-area unit. Alternatively, the anchor assembly can comprise a plurality of (e.g., two) high-surface-area anchor members and one or more connectors. The high-surface-area anchor member or the connecting member can be delivered via a needle. The high-surface-area anchor member can be coil-shaped. The shape of the high surface area anchor members may be a coil, deformable and autolocking, or barbed. An anchor assembly in accordance with the invention may be unitary or constituted of two or more pieces. If unitary, the anchor assembly may include a tensioning device to allocate more of the joining force to the ends of the anchor embers distal to the rupture.

On the other hand, in accordance,with the system, method, apparatus and other embodiments of the invention, a helical-based anchoring suture is connected (to another part of the ruptured tendon) across the site of tenorrhaphy by loops of other suture. The suture materials are delivered via needles. In such embodiments, a helical needle can be used to deploy the helical based anchoring suture and one or multiple straight needles can be used to deploy the loop-based connecting suture. The loop-based connecting suture can be shortened by multiple steps of length reduction for instance, via a knot.

In yet other good embodiments, the helical-based suture material is barbed. In various preferred embodiments, the suture members (and accordingly the constituent suture material) are absorbable. In various other embodiments, the connecting suture comprises two parts which join together between the cut ends of the tendon. The two parts can join together in a zip-tie fashion, or with a knot.

The various members and elements of the inventive system can comprise materials which are polymers. Preferably, the polymers are synthetic. The polymers can be nylon, polypropylene, polyethylene, polyester, polybutester, or any mixture of two or more thereof and preferably are absorbable. More specifically, the polymers are any of polylactic acid (PLA), polyglycolic acid (PGA), polycaprolactone (PGA), polydioxanone (PDX), or any mixture of two or more thereof. At least one of the monomers comprising the polymer may be enantiomerically pure.

In various additional good embodiments, a tool, grips an end of the ruptured tendon, such that one or more suture patterns can be deployed. The tool may be hand- or motor-driven, so as to effect deployment of the patterned suture. The drive of the needle assembly is achieved by a slider or a gear. The needle assembly drives the suture beyond the tendon surface proximal to the acute injury site. Optionally, the suture can be trimmed at the tendon surface proximal to the acute injury site.

The invention provides an apparatus and method for repair, recession, and revision of hand and wrist tendon ruptures that is completely internal, strong, flexible, and remodeled to simulate or even replicate the original orthopedic structure. This will increase repaired tendon strength, increase favorable patient outcome, reduce surgical procedure time, or allow earlier tendon flexion after surgery. In some embodiments, the apparatus includes a gripper, such as a clamp, holding the end of tendon proximal to the rupture. Such facilitation of alignment allows for the use of optimum sized fasteners without unnecessary or undue size increase. In some embodiments multiple suture patterns are inserted to stabilize the site of tenorrhaphy against twisting forces.

In accordance with certain embodiments of the invention, the apparatus can be used to deliver and implant the suture patterns in a particularly advantageous manner. A motor driven tool may insert the suture patterns into the ruptured surfaces of the tendon proximal to the site of acute injury. The tool may optionally hold the tendon secure proximal to the site of acute injury. The tool may optionally include a removable positioner that facilitates deployment of the suture patterns.

The implanted system or assembly is fabricated from polymeric materials. The polymers may be either natural or synthetic. If synthetic, the polymers may optionally be absorbable, such that high strength across the rupture is maintained for at least 6 weeks.

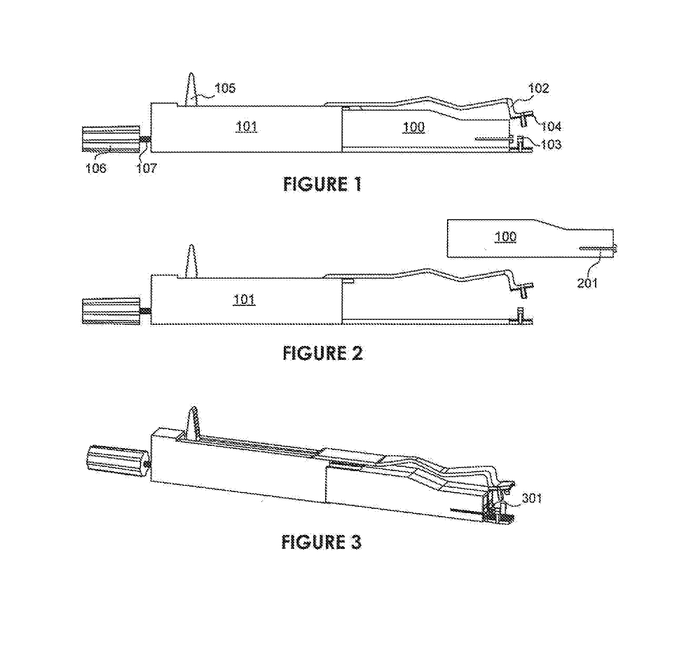

FIG. 1 provides a side view of apparatus in accordance with an embodiment of the invention. The assembly includes two types of needles for deploying the suture material within the tendon. (Typical needle elements and suture components are depicted in subsequent figures.) The deployment is facilitated by a clamp 102 that holds the tendon during the insert and removal of the needles. The clamp has a gripping surface 104 to hold the tendon and locking mechanism 103 to keep the clamp closed An actuator with an external drive knob 106, leadscrew 107, and slider knob 105 drives the deployment of the needles and suture. The apparatus includes a reusable part 101 and disposable cartridge 100 and a reusable part 101 on which the cartridge is mounted.

FIG. 2 provides a side view of an embodiment of the invention. In this embodiment, the needles and suture are within the disposable cartridge 100. The disposable cartridge, shown separately from the reusable part 101, attached to the reusable part by a detent or other type of connector. The disposable cartridge contains details 201 to facilitate management of the suture.

FIG. 3 provides an axonometric view of an assembly in which the disposable needle parts are located within the tool body in accordance with an embodiment of the invention. A gate 301 facilitates alignment of the straight needles, helical needles, and tendon during insertion of the patterns of suture.

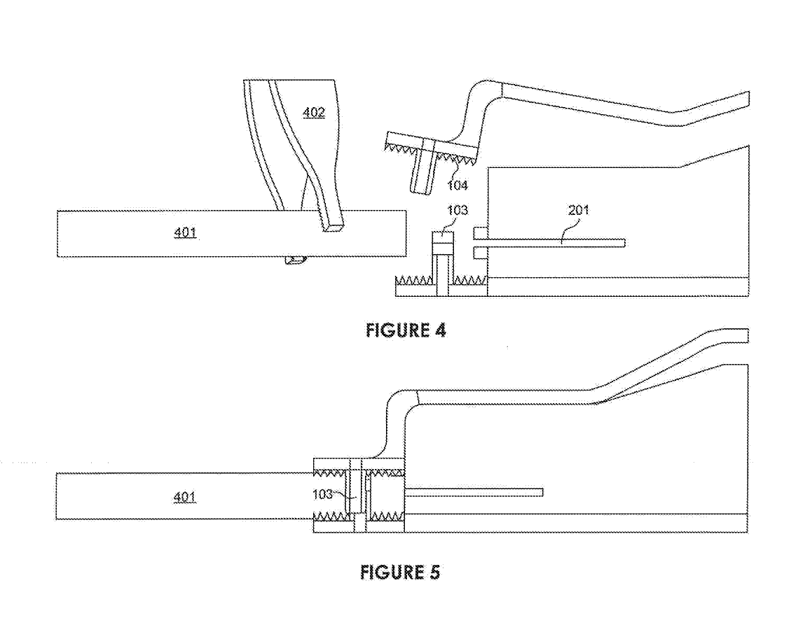

FIG. 4 provides a side view of forceps holding a ruptured tendon, and a clamp comprising elements 103 and 104, within the apparatus. The clamp facilitates the alignment of the needles with the tendon 401 so that the suture patterns are deployed nearly parallel to the long axis of the tendon by the actuator. Forceps 402 can be used to retain and insert the tendon into the device. FIG. 5 provides a side view of the clamp holding the tendon 401 firmly for insertion of needles and suture. This system is completely internal to the tendon, has high surface area, and maintains a less than 2 mm gap, between the tendon surfaces for at least 6 weeks, until the tendon has healed to sufficient tensile strength.

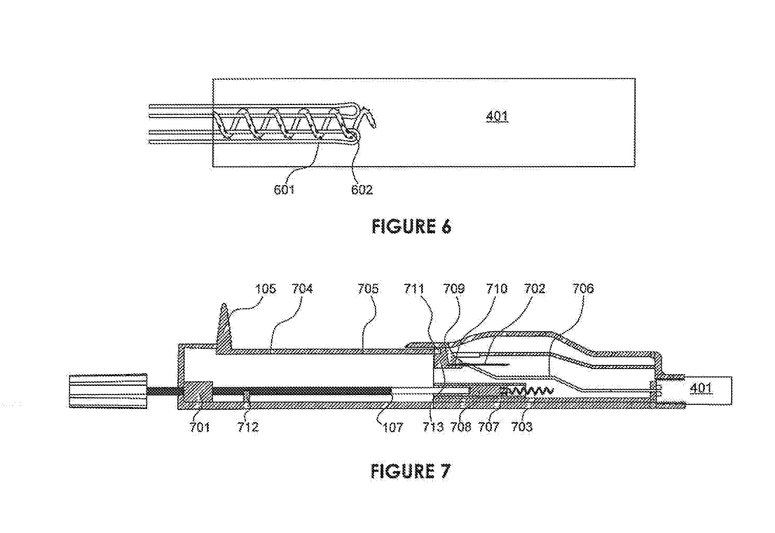

In a preferred embodiment, two helical patterns of barbed suture and four loop patterns of non-barbed suture are deployed simultaneously into one of the two cut surfaces of tendon proximal to the site of rupture. FIG. 6 provides a transparent top view of an interlocking suture pattern deployed within the tendon in accordance with an embodiment of the invention. Both the loop suture 601 and the helical suture 602 are shown affixed in the tendon 401. FIG. 7 provides a section view of straight and helical needles 702 and 703, respectively; contained within the apparatus in accordance with this embodiment of the invention. The slider 704 drives the, straight needles 702. Pins 705 and 709 guide the slider in a track 706 in the disposable cartridges The slider head pin 709 also supports the needle during insertion. The slider 704 its into the slider head 713 via a slot 711. The straight needle fits into the slider head via a slot 710. The leadscrew is engaged by a nut 701 held in the reusable tool. A nut stop 712 allows for sliding motion of the leadscrew to engage the gate and tendon 401. The leadscrew drives the helical needle 703. The leadscrew is attached to a tube 708 that attaches to the helical needle 703. The base of the helical needle has a very fine pitch 707 to facilitate attachment to the tube 708.

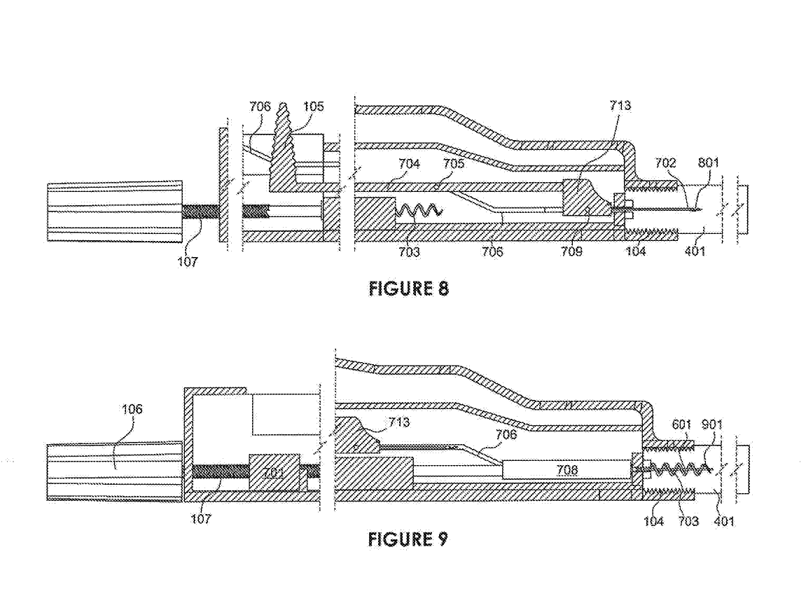

FIG. 8 provides a section view of straight needles and suture in the tendon at a point midway through the deployment process in accordance with an embodiment of the invention. The straight needle 702 contains a notch 801 that retains the suture during insertion. FIG. 9 provides a section view of helical needles and suture. The helical needles that deploy the helical pattern of suture must insert the suture material arid leave the suture material in place as the needles are removed. FIG. 9 shows a helical needle having a groove and a hole adapted to hold barbed suture, in the tendon 401 at a point midway through the deployment process in accordance with an embodiment of the invention. There is a groove in which the suture material sits during insertion into the tendon. There is a hole 901 at the tip of the needle that is adapted to hold suture during insertion and to release the suture as the needle is withdrawn. The suture retained by the helical needle engages the loop suture 601 previously inserted by the straight needles into the tendon 401.

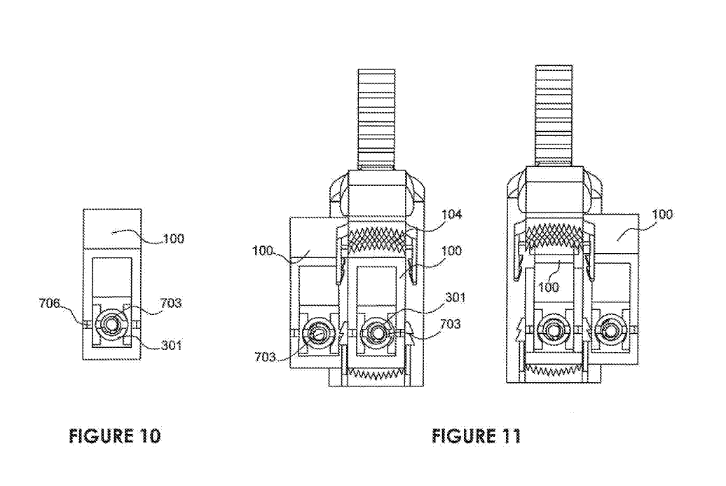

The delivery of the suture by the helical needle engages the hops of suture delivered by the straight needles. Precise alignment of the straight;and helical needles facilitates this delivery of the interlocking suture patterns. FIGS. 10 provides a view of a gate sub-assembly 301 adapted to facilitate the alignment of the straight needles and helical needle 703, in accordance with an embodiment of the invention.

To achieve tenorrhaphy, patterns of helical and loop suture are inserted first into one severed end of the tendon proximal to the site of injury and then into the other severed end of the tendon proximal to the site of injury. Delivery of the patterns into both ends of the severed tendon requires two helical needles 703, four straight needles 702, and two types of suture. FIG. 11 provides a front view of apparatus wherein a first set of disposable needle parts within the tool body (left-hand image), and of second set of disposable needle parts (right-hand image) after a shift within the tool body in accordance with an embodiment of the invention.

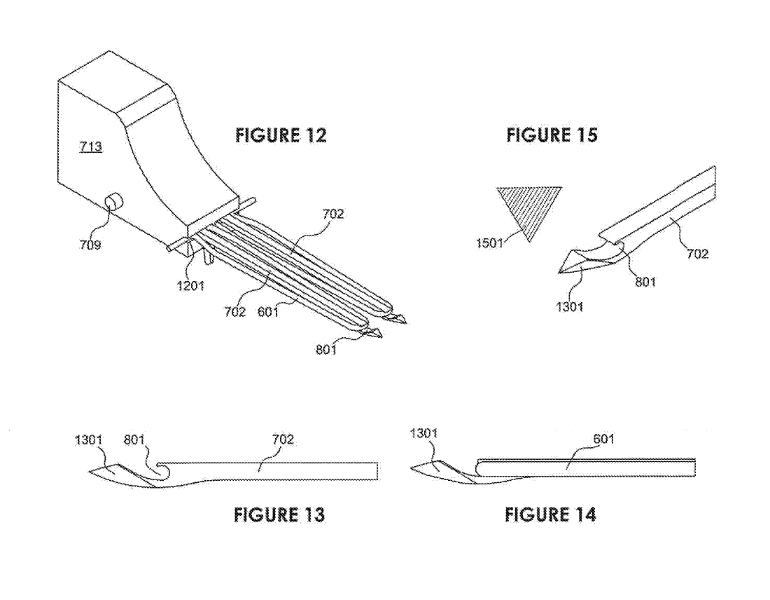

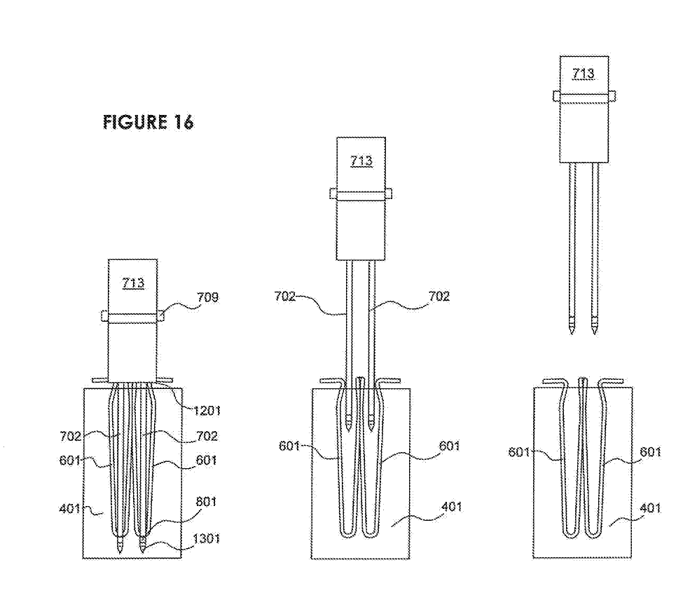

The straight needles that deploy the loop pattern of suture insert the suture material and leave the suture material in place as the needles are removed. FIG. 12 provides an axonometric view of a pair of straight needles attached to a sliding member in accordance with an embodiment of the invention. The sliding member is used to drive the straight needles 702 and loop suture 601 into the severed end of the tendon proximal to the site of injury. The suture 601 routes through a suture management feature 1201 in the slider head 713. FIG. 13 provides a side view of a straight needle 702 having a notch 801 adapted to hold suture in accordance with an embodiment of the invention. The needle tip 1301 is sharpened. FIG. 14 provides a side view of loop suture 601 engaged in a notch of a straight needle in accordance with an embodiment of the invention. Sharpness of the needle tip 1301 facilitates insertion into the tendon FIG. 15 provides axonometric views of the sharpened tip 1301 and notch 801 of a straight needle 702 in accordance with an embodiment of the invention. The straight needle tip profile 1501 is triangular in section view. FIG. 15 provides a top view of straight needles 702 having notches 801 adapted to hold suture 601 and suture 601 within the tendon at various stages as the needles are withdrawn in accordance with an embodiment of the invention. The suture 601 that remains n the tendon 401 forms a loop in this embodiment of the invention. The suture management feature 1201 facilitates the release of the suture from the slider head 713.

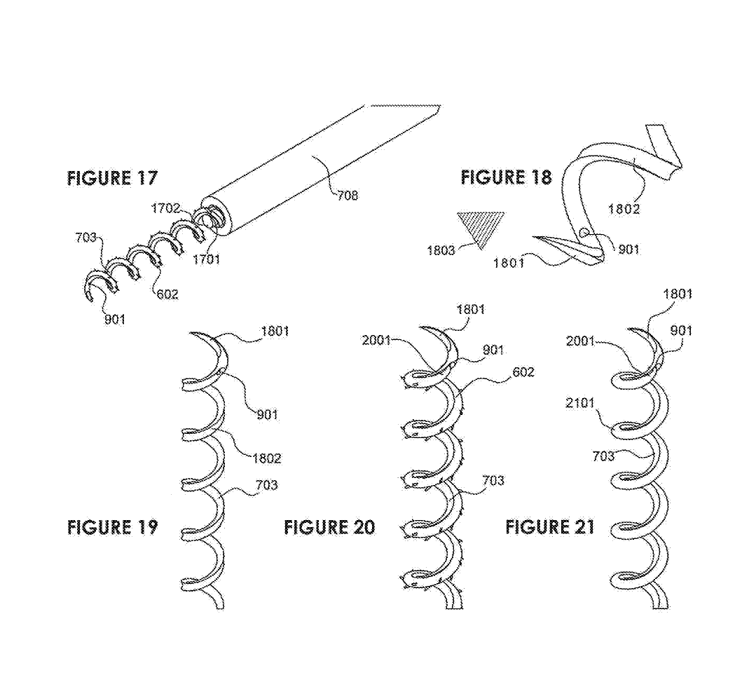

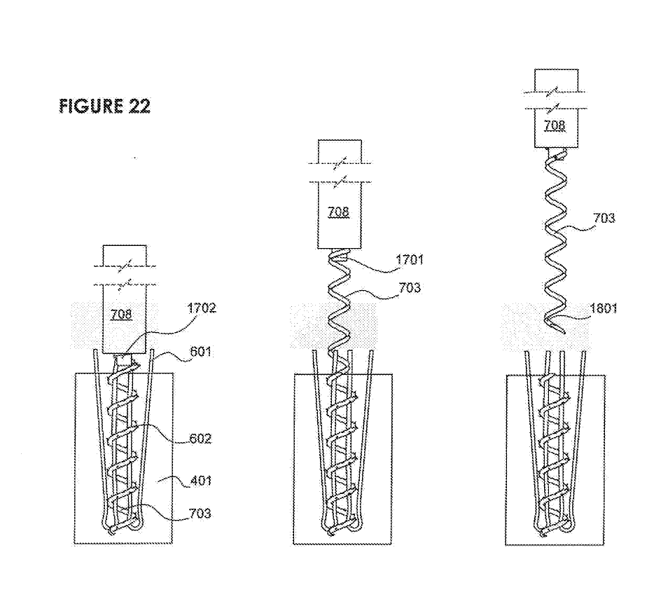

The helical needle that deploys the helical pattern of suture must insert the suture material and leave the suture material in place as the needle is removed. FIG. 17 provides an axonometric view of a helical needle 703 attached to the tube 708 in accordance with an embodiment of the invention. The helical needle is attached to the leadscrew by a tube 708 and spindle 1702 in one embodiment of the invention. The helical needle contains a hole near the tip 901 and near the base 1701 that facilitate retention of the suture 602 during insertion. FIG. 18 provides an axonometric view of a helical needle having a groove 1802 and hole 901 adapted to retain suture and a section view of the sharpened tip in accordance with an embodiment of the invention. A sharpened tip 1801 on the helical needle facilitates insertion of the needle and suture. The needle tip profile 1803 is triangular, reverse-cutting. FIG. 19 provides axonometric views of the sharpened reverse-cutting tip 1801 and groove 1802 of the helical needle 703 in accordance with an embodiment of the invention. In one embodiment of the invention, a hole 901 near the tip of the helical needle retains the suture during insertion. FIG. 20 provides an axonometric view of barbed suture 602 engaged with a helical needle 703 having a groove and hole 901 adapted to retain suture in accordance with an embodiment of the invention. The end of the helical suture 2001 distal to the site of injury in the tendon is shaped to fit within the hole 901. In another embodiment of the invention, the helical needle can insert smooth suture. FIG. 21 provides an axonometric view of smooth suture 2101 engaged, with a helical needle 703 having a groove and hole 901 adapted to retain suture in accordance with an embodiment of the invention. When the helical needle is withdrawn, the suture remains in a helical pattern in the tendon. FIG. 22 provides a transparent top view of a helical needle 703 and suture 602 that remains within the tendon 401 at various stages as the helical needle is withdrawn in accordance with an embodiment of the invention. The helical suture 602 has engaged with the loop suture 601.

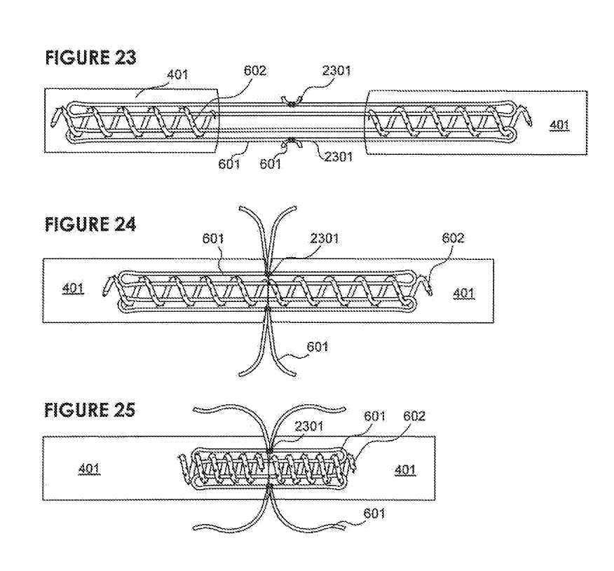

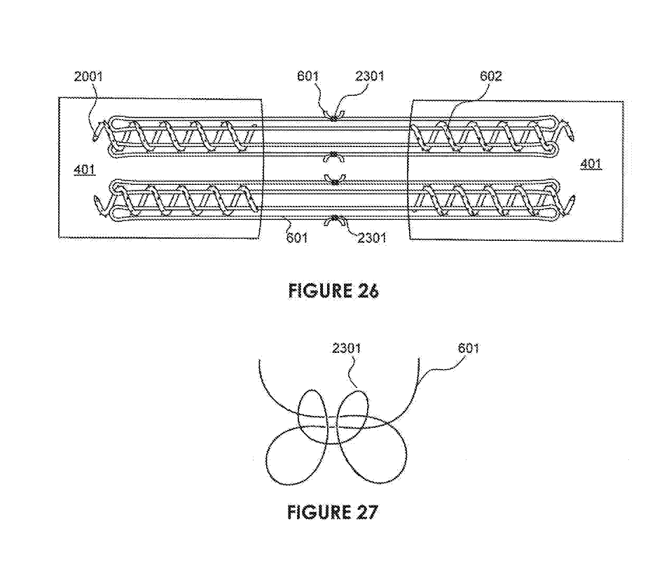

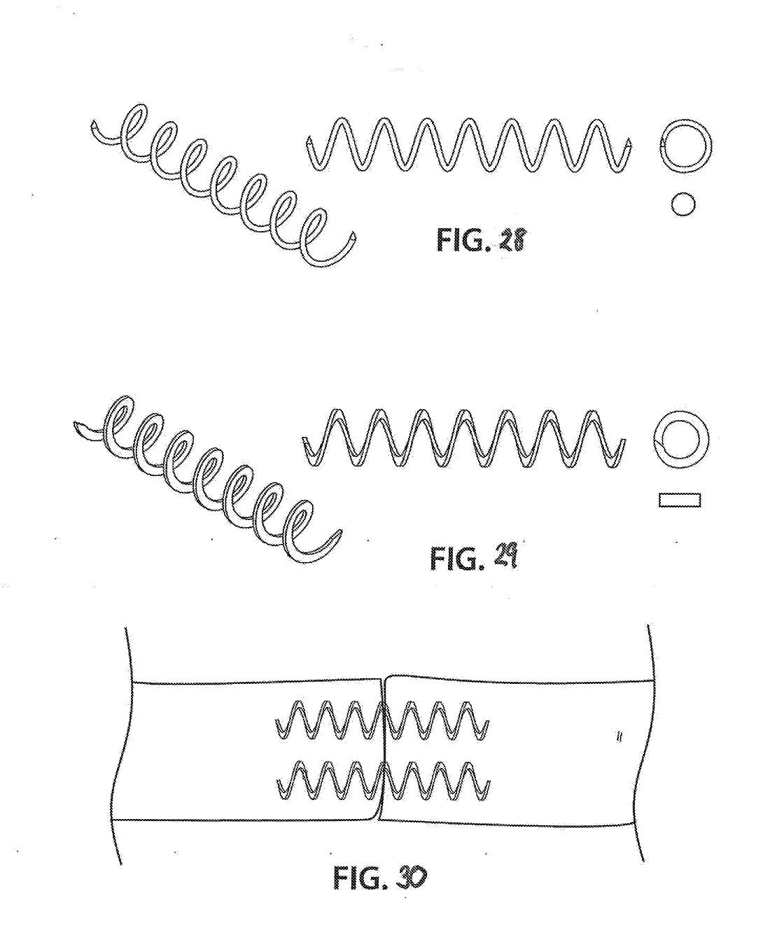

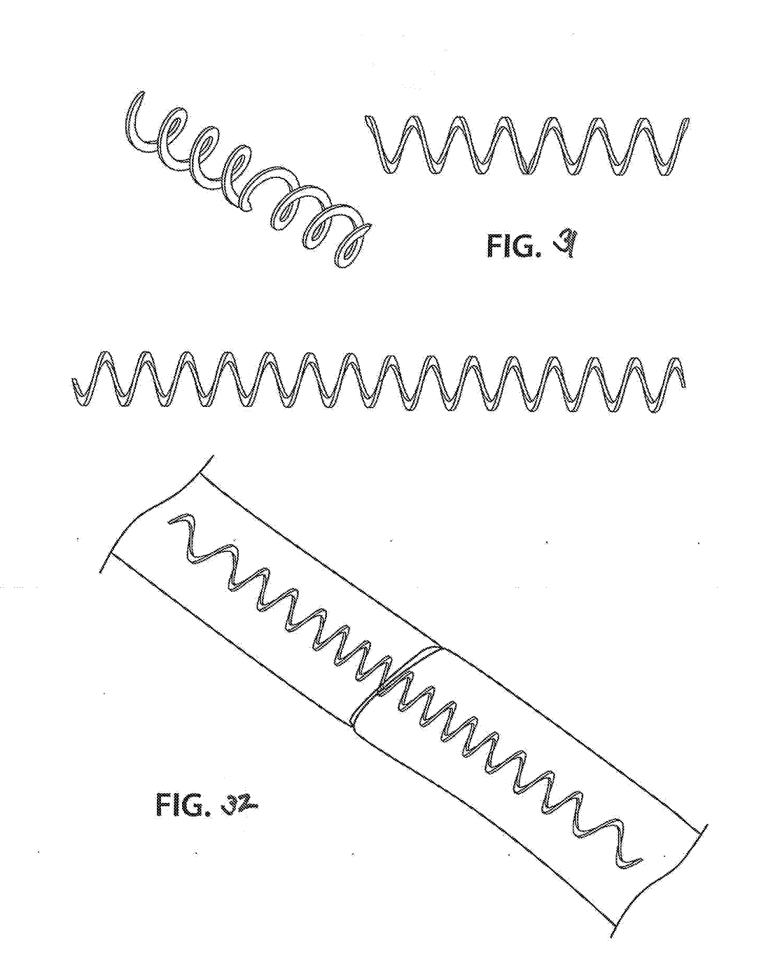

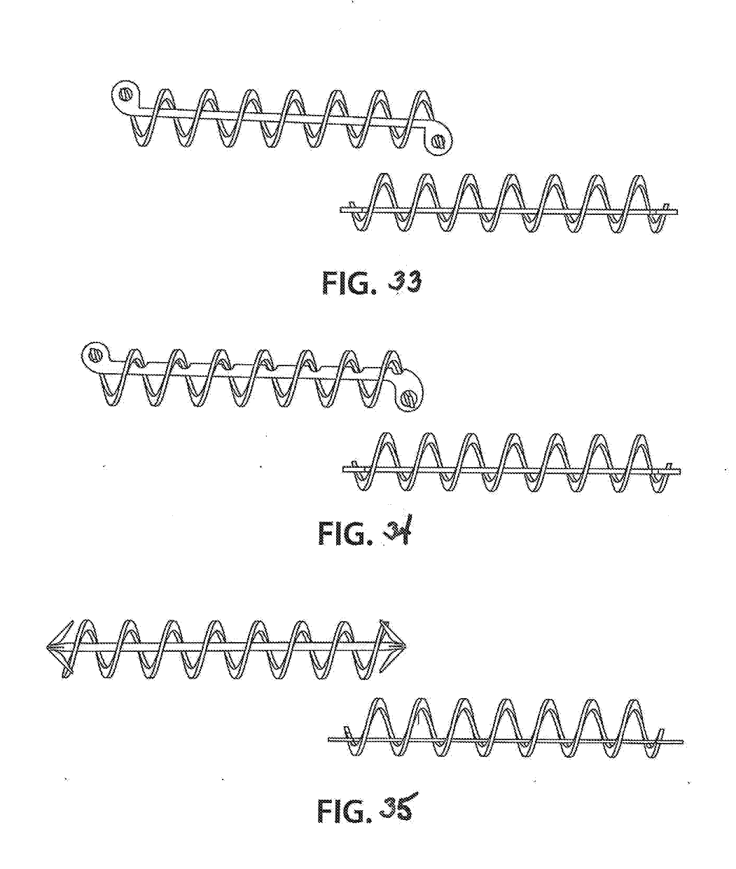

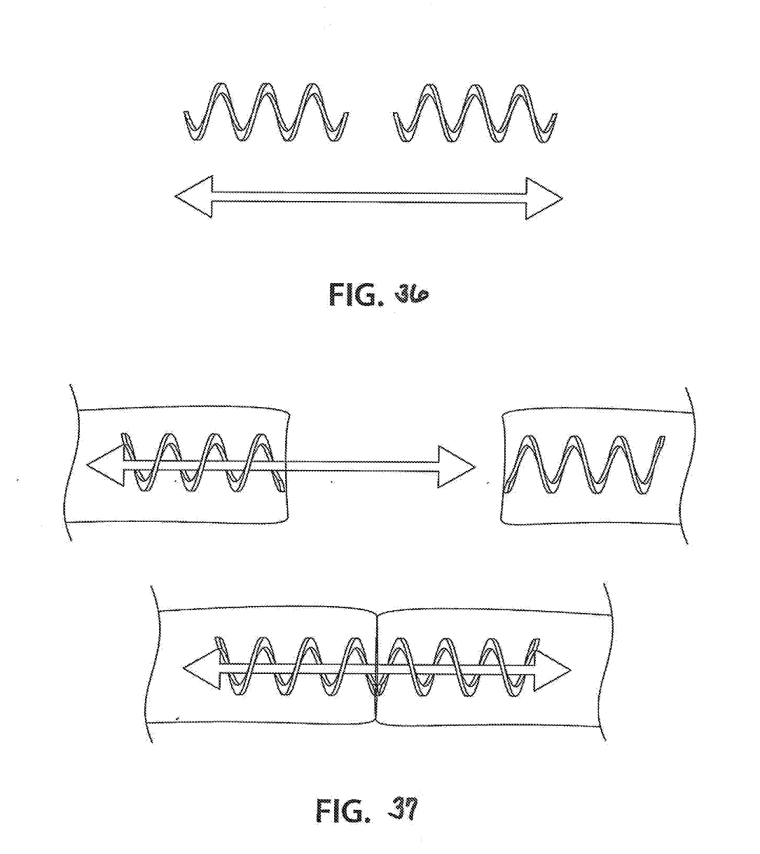

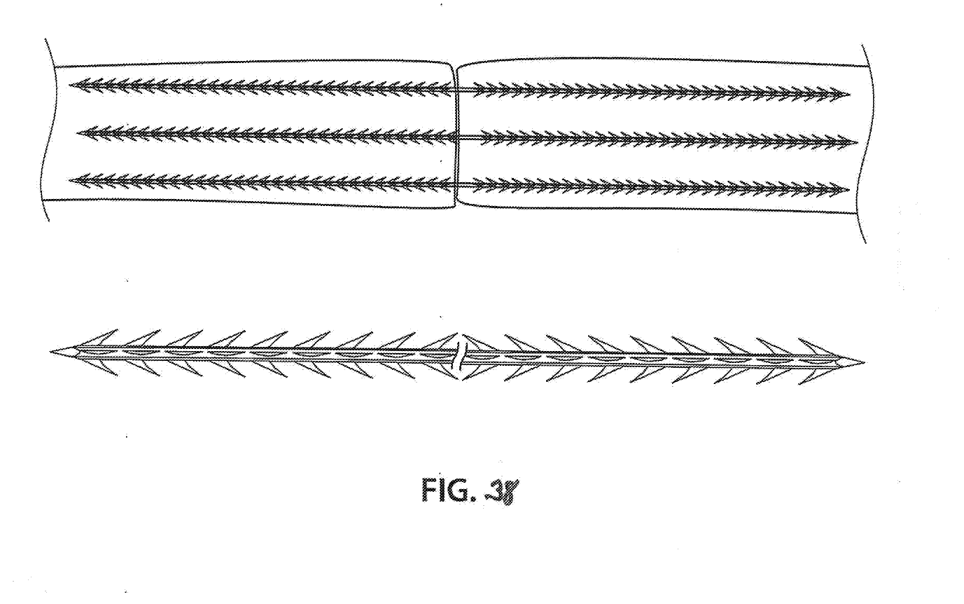

In certain advantageous embodiments of the invention helical and loop patterns of suture have an interlocking configuration. In various embodiments of the invention, the connector element(s), such as sutures, provide a tensioning influence on the system securing the ruptured tendon ends. FIG. 23 provides a transparent top view of interlocking suture patterns deployed within the tendon 401 to facilitate tenorrhaphy in accordance with an embodiment of the invention. In this embodiment, the loop suture 601 physically captures the top coils of the helical suture 602. The same patterns of suture are then deployed to the other end of the tendon proximal to the site of rupture. The one or multiple pairs of loop suture 601 material must be joined. This can be accomplished with a knot 2301. The knot 2301 can be pre-tied before the suture is deployed. After the suture patterns are inserted into both ends of the tendon adjacent to the site of injury, the gap between the two pieces of tendon must be reduced. FIG. 24 provides a transparent top view of interlocking suture patterns deployed within the tendon, fastened and tightened across the site of tenorrhaphy with multiple knots 2301 to adjoin the tendon 401 ends, in accordance with an embodiment of the invention. The loop patterns of suture are tightened and secured across the site of tenorrhaphy, possibly in multiple steps to firmly interlock the loops with the helices. FIG. 25 provides a transparent top view of suture fastened across the site of tenorrhaphy to approximate the severed tendon 401 in which the interlocking pattern has been set, in accordance with an embodiment of the invention. Additional steps of length reduction set the knot 2301 and fully engage the interlocking patterns of suture 601 and 602. This pattern distributes the tensile force holding the tenorrhaphy together roughly 1 cm distal to the site of injury. Multiple helical patterns may be deployed within each side of the tendon adjacent to the site of injury. Two patterns of suture repair provide resistance to twisting forces on the tendon. FIG. 26 provides a transparent top view of two instances of loop 601 and helical suture pattern 602 repairs fastened across the site of tenorrhaphy to approximate the severed tendon 401 ends in accordance with an embodiment of the invention. FIG. 27 provides a top view of a knot 2301 operative to facilitate the adjustment of the length of the loop suture 601 in accordance with an embodiment of the invention. The knot can tighten when the ends of the suture are pulled and the gap between the tendon ends is reduced. The knot is low volume, which aids remodeling of the tendon to the original, uninjured biological structure.

FIG. 28 is four views of a coil-type high-friction member including the profile shape of the material used.

FIG. 29 is an optimized version of FIG. 28 with an elongated profile shape.

FIG. 30 demonstrates the placement of two single-piece coil anchors within the tendon.

FIG. 31 is another version of the coil-type member, being made in one piece of both clockwise and counterclockwise coils.

FIG. 32 is a variable-rate coil-type member shown both at rest anal under longitudinal force within the tendon, displaying increased stiffness at site proximal to tendon rupture.

FIGS. 33-35 are coil-type members displaying various tensioners which prevent the coils from elongating, including two-hole, hole-and-notch, and expanding barb (shown side view).

FIG. 36 depicts two coil-type high-friction members and a barbed connecting member, which would be inserted into the members, which together form a single anchor.

FIG. 37 shows one end of the connecting member installed within a coil-type member on one side of the ruptured tendon, as well as a view of the completed repair.

FIG. 28 illustrates a coil-type high-surface-area anchor. As will be further described herein the anchor is adapted to facilitate the joining of two ends of a tendon proximal to the site of rupture. The anchor is inserted into the end of the tendon proximal to the site of rupture, providing a strong, flexible, internal joint when a connection is also made to the end of the tendon on the other side of the rupture. FIG. 29 illustrates a coil whose cross section is elongated in the direction perpendicular to the axis of the coil. This elongation provides additional surface and frictional force in the direction along the tendon major axis. Multiple anchors can be inserted into a single ruptured tendon. In the embodiment of FIG. 30, two single coils are inserted to join the two ends of a ruptured tendon proximal to the site of the acute injury. In the preferred embodiment of FIG. 31, a single coil of opposing helicities is used to join the two ends of a ruptured tendon proximal to the site of the acute injury.

There are various methods to insert the coil anchor into the end of the tendon proximal to the site of rupture. FIG. 32 illustrates a variable rate coil, which provides for increased tension at the ends of the anchor distal to the site of tendon rupture. Alternatively, increased tension can be provided to the middle of the coil. Said variable rate coil is an auto-tensioning device. FIGS. 33-35 illustrate other types of tensioners that provide for increased tension to the coil distal to the site of tendon rupture, including two-hole, hole-and-notch, and expanding barb. FIG. 36 shows a barbed tensioner to be inserted into a dual coil anchor system with opposing coil helicities. FIG. 37 shows insertion of two coils of opposite helicity into the two ends of the tendon proximal to the rupture and connected, with the connector also serving as a tensioner.

FIG. 38 shows a high surface area attachment, where multiple barbs provide the engagement force on the tendon. FIG. 38 shows in particular, a single-piece anchor with barbs angled to provide engagement force in opposite directions on the two pieces of tendon proximal to the site of rupture.

### EXAMPLES

In a preferred embodiment, two helically patterned barbed suture elements and four loop patterned non-barbed suture elements are deployed simultaneously to one of the surfaces of the tendon proximal to the site of rupture. The same patterned suture elements are then deployed to the other surface of the tendon proximal to the site of rupture. The tool employed includes clamps that hold the tendon and facilitate alignment of the needles used to deploy the patterned suture elements. The loop patterned suture elements are then tightened and secured across the site of tenorrhaphy, possibly in multiple steps to firmly interlock the loops with the helices. This system is completely internal to the tendon, is flexible, and maintains a less than 2 mm gap between the tendon surfaces for at least 6 weeks, until the tendon has healed to sufficient tensile strength.

In a second preferred embodiment, innovative apparatus of the invention facilitates firm interlocking of the loop and helical suture patterns as the length of the suture connection across the site of tenorrhaphy is reduced. The tool includes clamps that affix to the two severed ends of the tendon. These clamps provide a resisting force during the process of tightening the suture. This resisting force facilitates settling of the suture material in the tendon and firm interlocking of the loop and helical suture patterns. This system is completely internal to the tendon, has high surface area, and maintains a less than tram gap between the tendon surfaces for at least 6 weeks, until the tendon has healed to sufficient tensile strength.

Numerous characteristics and advantages of the invention covered by this document have been set forth in the foregoing description. It will be understood, however, that this disclosure is, in, many respects, only illustrative. Changes can be made in details, particularly in matters of shape, size, and ordering of steps without exceeding the scope of this invention. The invention's scope is, of course, defined in the language in which the appended claims are expressed.

What is claimed is:

1. A system for tenorrhaphy, which comprises a plurality of high surface area anchor members, each said member being operative for implantation in a part of the tendon proximal the rupture, and each said member further being adapted for interconnection with another of said members also operative for implantation in a part of the tendon proximal the rupture; and a connector for attaching one of said members to one another of said members.

2. A method for tenorrhaphy, which comprises implanting a plurality of high surface area anchor members in respective different parts of the tendon proximal the rupture; and interconnecting one of said members with another of said members.

3. An apparatus for implanting a repair assembly including a plurality of high surface area anchor members into a ruptured portion of a tendon, which comprises a needle assembly for implanting an anchor member as aforesaid in a part of the tendon proximal said rupture; and a mechanism for introducing a connector for attaching said anchor member to another anchor member.

4. An apparatus for tenorrhaphy, which comprises
multiple solid needles of two or more different types, operative to attach a suture to a proximal cut surface of a tendon, and an actuator for the needle,
an element to secure the alignment of said needles with the cut end of the tendon,
said needles being configured to deploy said suture in an interlocking pattern,
whereby said suture is presented for joining, with another end of the ruptured tendon across the site of tenorrhaphy, and
an element for manipulating said needles to deploy said suture in the cut tendon end.

5. A method for tenorrhaphy which comprises
aligning a plurality of needles capable of deploying a suture to a severed end of cut tendon,
manipulating said needles to deploy said suture in the cut tendon surface in an interlocking pattern, and to present said suture for joining with another cut tendon end at the site of tenorrhaphy.

6. An apparatus for tenorrhaphy for inserting suture in interlocking patterns, which comprises a compliment of solid needles of o or more different types for inserting suture, and a mechanism for alignment of said needles with the severed ends of the tendon,
whereby said two severed ends of the tendon at the site of tenorrhaphy are joined via said suture,
whereby said suture ends are fastened across the site of tenorrhaphy.

7. An apparatus according to claim 4, wherein said needles include features adapted to hold said suture.

8. An apparatus according to claim 4, wherein said needles comprise helical and straight needles adapted to insert said suture at the site of tenorrhaphy.

9. An apparatus according to claim 8, wherein the insertable parts of said straight needles are less than 20 mm in length and less than 1.2 mm in the transverse dimensions.

10. An apparatus according to claim 8, wherein the insertable parts of, said straight needles are less than 20 mm in length and less than 1.0 mm in the transverse dimensions.

11. An apparatus according to claim 8, wherein the insertable parts of said helical needles are less than 20 mm in length and less than 5 mm in helical diameter, having a pitch between 1 mm and 4 mm, and being of wire gauge less than 0.8 mm.

12. An apparatus according to claim 8, wherein the insertable parts of said helical needles are less than 20 mm in length and less than 4 mm in helical diameter, having a pitch between 1 mm and 3 mm, and being of wire gauge less than 0.6 mm.

13. An apparatus according to claim 4, wherein said suture is between 2-0 and 4-0 USP, inclusive, and including maximum oversize.

14. An apparatus according to claim 8, wherein said helical needles have cutting or reverse cutting suture needle points and wherein said straight needles have sharpened points.

15. An apparatus according to claim 4, wherein at least one type of said needles includes a groove or notch in which the suture lies and features adapted to attach said suture to the needles and to allow release of said suture during deployment.

16. An apparatus according to claim 4, wherein at least one type of said needles includes a groove in which the suture sits and one or greater holes adapted to attach said suture to the needles and to allow release of said suture during deployment.

17. An apparatus according to claim 4, wherein said interlocking patterns comprise loop parts and helical parts, the loop parts adapted to interlock with the helical parts.

18. An apparatus according to claim 4, wherein said needles can be manipulated to reduce the length of sad suture to set the interlocking pattern and optionally to facilitate approximation of the two tendon ends at the site of tenorrhaphy.

19. An apparatus according to claim 4, wherein said needles can be manipulated multiple interlocking patterns.

20. An apparatus according to claim 4, whereby said needles and suture are deployed without breach of intact epitenon.

21. An apparatus according to claim 4, whereby said suture is knotted to cause the adjustment of the length of said suture across the site of tenorrhaphy.

22. An apparatus according to claim 21, whereby said adjustment comprises length reduction of said suture to facilitate approximation of the two tendon ends at the site of tenorrhaphy.

23. An apparatus according to claim 4, which is adapted to deploy suture of a synthetic polymer or polymers.

24. An apparatus according to claim 23, which is adapted to deploy suture that is barbed.

25. An apparatus according to claim 23, which is adapted to deploy suture that is absorbable.

26. An apparatus according to claim 24, which is adapted to deploy suture that is absorbable.

27. A system according to claim 1, additionally comprising chemical or biological agents to prevent adhesion, prevent infection, or aid healing.

28. An apparatus according to claim 4, which further comprises one or more sliding members to actuate at least one type of said needles.

29. An apparatus according to claim 28, wherein said sliding member includes features to manage suture delivery and release.

30. An apparatus according to claim 4, which further comprises one or more leadscrews and nuts to actuate at least one type of said needles.

31. An apparatus according to claim 4, which further comprises an element through which said two types of needles pass and whereby facilitates alignment of said needles to the tendon.

32. An apparatus according to claim 4, which further comprises one or more clamps that hold and align said severed end of tendon for engagement with said needles.

33. An apparatus according to claim 4, wherein a tool body facilitates actuation of one or greater sliding members that actuate at least one type of said needles, one or greater leadscrews and nuts that actuate at least one type of said needles, a feature through which said two types of needles pass and that facilitates alignment of said needles to the tendon, and one or greater clamps that hold and align said severed end of tendon for engagement with said needles.

34. An apparatus according to claim 4, herein said apparatus includes reusable parts and disposable parts.
